(19) United States
(12) Patent Application Publication
(10) Pub. No.: US 2025/0194927 A1
(43) Pub. Date: Jun. 19, 2025

(54) SYSTEM AND METHOD OF DETECTING MELANOMA ON A PATIENTS SKIN

(71) Applicant: Ramzor Diagnostic Ltd, Netivot (IL)

(72) Inventors: Dmitry LIHOVETSKI, Beer Sheva (IL); Boris MELNIK, Netivot (IL)

(73) Assignee: Ramzor Diagnostic Ltd, Netivot (IL)

(21) Appl. No.: 18/539,330

(22) Filed: Dec. 14, 2023

(51) Int. Cl. A61B 5/00; G16H 50/20

(52) U.S. Cl. CPC A61B 5/0064; A61B 5/444; G16H 50/20

(57) ABSTRACT

Systems and methods for detection of melanoma on a patient's skin, including: a detector, configured to capture signals that are scattered of the skin, a rail, configured to allow movement of the detector on the rail in two perpendicular axes at a resolution of 0.5 millimeters, and a processor, coupled to the detector and configured to: receive signals captured by the detector, map the received signal to the position of the detector on the rail, compare data from each point with a predetermined melanoma threshold, identify at least one spread of melanoma pattern based on the comparison and the mapping, and determine if the skin includes melanoma, based on the results of the comparison, and based on identification of the at least one spread of melanoma pattern.

### FIELD OF THE INVENTION

The present invention relates to non-invasive monitoring of patients. More particularly, the present invention relates to systems and methods for detection of melanoma on a patient's skin.

### BACKGROUND OF THE INVENTION

Melanoma is well-investigated malignant skin tumor that emerges and develops in subcutaneous layers at the border of the epidermis and dermis. At the initial stages of the decease, there are slow changes in the cell generation, cell geometry, nucleus to plasma ratio, division rate, etc. The growth of melanoma is accompanied by a vast generation of non-standard melanocytes that take the place of the standard ones, leaving no room for the cytoplasm and keratinocytes. The non-standard melanocytes accumulate a large amount of melatonin, a pigment that is untypical for a healthy tissue.

In the later stages, malignant cells spread through the skin, and melanocytes become dominant. Since the melanin creates pigmentation, melanoma acquires a variety of colors that can be detected by visual observation, for example, using dermatoscopy. Oftentimes, visual observability of melanoma becomes feasible only when treatment is already challenging or is no longer possible.

Since the densities of malignant and of healthy tissues are approximately the same, X-ray based methods, such as CT scanning, are uncapable of detecting the tumor, when the latter is still hidden in the subcutaneous skin layer.

### SUMMARY OF THE INVENTION

There is thus provided, in accordance with some embodiments of the invention, a system for detection of melanoma on a patient's skin, the system including: a detector, configured to capture signals that are scattered of the skin; a rail, configured to allow movement of the detector on the rail in two perpendicular axes at a resolution of 0.5 millimeters; and a processor, coupled to the detector and configured to: receive signals captured by the detector, wherein each received signal includes NxM points, wherein the detector collects ‘D’ samples to get a three-dimensional output of NxMxD, where ‘N’, ‘M’ and ‘D’ are integer numbers, map the received signal to the position of the detector on the rail, compare data from each point with a predetermined melanoma threshold, identify at least one spread of melanoma pattern based on the comparison and the mapping, and determine if the skin includes melanoma, based on the results of the comparison, and based on identification of the at least one spread of melanoma pattern.

In some embodiments, the rail is a mechanical rail, and the detector is moved mechanically. In some embodiments, the rail includes a plurality of movable mirrors, wherein each mirror is movable between a first state that is parallel to the rail surface and a second state that is rotated by an angle as compared to the first state, and wherein the second state allows receiving and sending signals to the skin. In some embodiments, a plurality of sources irradiates the skin, such that the detector receives signals from scattered irradiation.

In some embodiments, the detector includes a spectroscope and a dermatoscope, and wherein the processor is further configured to perform image processing on the received signal by the dermatoscope. In some embodiments, the processor is further configured to train a machine learning (ML) algorithm to predict a melanoma. In some embodiments, the processor is further configured to apply the ML algorithm on the received signals captured by the detector.

In some embodiments, the processor is further configured to apply clustering for input to the ML algorithm, wherein the clustering includes clustering on at least one of: patterns and spectrum responses for the received signals.

In some embodiments, the clustering includes clustering on spectrum analysis compared to a reference sample associated with melanoma probability exceeding the predetermined melanoma threshold. In some embodiments, the clustering includes clustering on spectrum analysis compared to a sample of a group of patients.

There is thus provided, in accordance with some embodiments of the invention, a method of detecting melanoma on a patient's skin, the method including: capturing, by a detector, signals that are scattered of the skin, moving the detector on a rail in one of two perpendicular axes at a resolution of 0.5 millimeters, and receiving signals captured by the detector, wherein each received signal includes NxM points, wherein the detector collects ‘D’ samples to get a three-dimensional output of NxMxD, where ‘N’, ‘M’ and ‘D’ are integer numbers, comparing data from each point with a predetermined melanoma threshold, mapping the received signal to the position of the detector on the rail, identifying at least one spread of melanoma pattern based on the comparison and the mapping, and determining if the skin includes melanoma, based on the results of the comparison, and based on identification of the at least one spread of melanoma pattern.

In some embodiments, the rail is a mechanical rail, and the detector is moved mechanically. In some embodiments, the rail includes a plurality of movable mirrors, wherein each mirror is movable between a first state that is parallel to the rail surface and a second state that is rotated by an angle as compared to the first state, and wherein the second state allows receiving and sending signals to the skin. In some embodiments, the skin is irradiated, by a plurality of sources, such that the detector receives signals from scattered irradiation.

In some embodiments, the detector includes a spectroscope and a dermatoscope, and the method further includes performing image processing on the received signal by the dermatoscope. In some embodiments, a machine learning (ML) algorithm is trained to predict a melanoma. In some embodiments, the ML algorithm is applied on the received signals captured by the detector.

In some embodiments, clustering is applied for input to the ML algorithm, wherein the clustering includes clustering on at least one of: patterns and spectrum responses for the received signals.

In some embodiments, the clustering includes clustering on spectrum analysis compared to a reference spectrum of a sample associated with melanoma probability exceeding the predetermined melanoma threshold. In some embodiments, the clustering includes clustering on spectrum analysis compared to a sample of a group of patients.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a block diagram of a computing device, according to some embodiments of the invention;

FIGS. 2A-2C schematically illustrates a system for detection of melanoma on a patient's skin, according to some embodiments of the invention;

FIG. 3A schematically illustrates a rail of a system for detection of melanoma 100 on a patient's skin with a mirror configuration, according to some embodiments of the invention;

FIG. 3B schematically illustrates a multiple source configuration of the system 350 for detection of melanoma 100 on a patient's skin 10 with a plurality of sources irradiating the skin, according to some embodiments of the invention; and

FIG. 4 shows a flowchart for a method of detecting melanoma on a patient's skin, according to some embodiments of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items.

Unless explicitly stated, the method embodiments described herein are not constrained to a particular order or sequence. Additionally, some of the described method embodiments or elements thereof may occur or be performed simultaneously, at the same point in time, or concurrently.

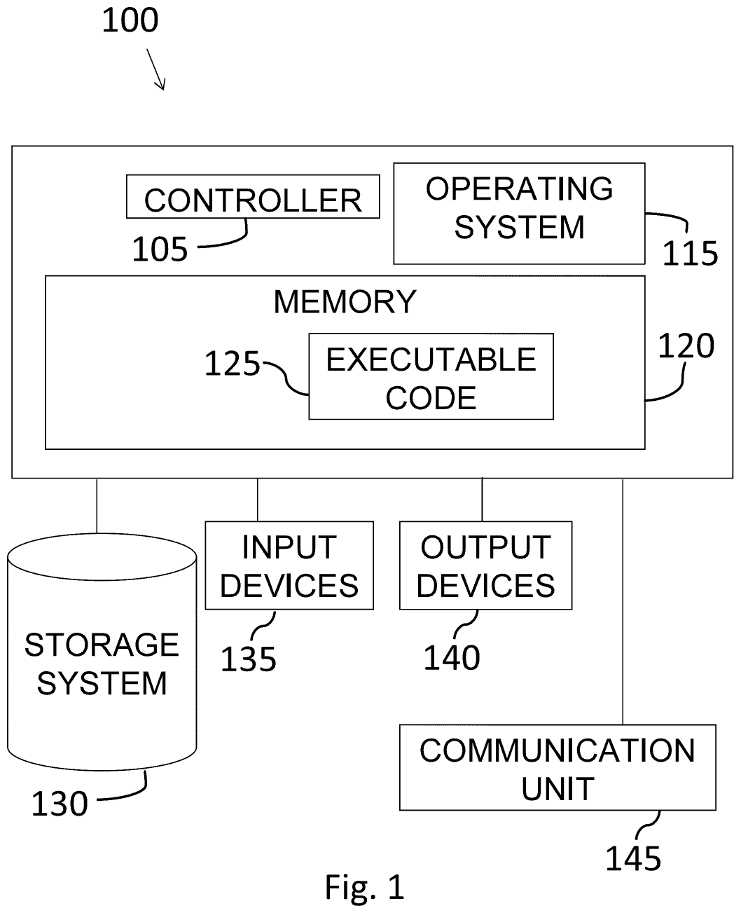

Reference is made to FIG. 1, which is a block diagram of an example computing device, according to some embodiments of the invention. Computing device 100 may be or may include a controller or processor 105 (e.g., a central processing unit processor (CPU), a chip or any suitable computing or computational device), an operating system 115, memory 120, executable code 125, storage 130, input devices 135 (e.g., a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet.

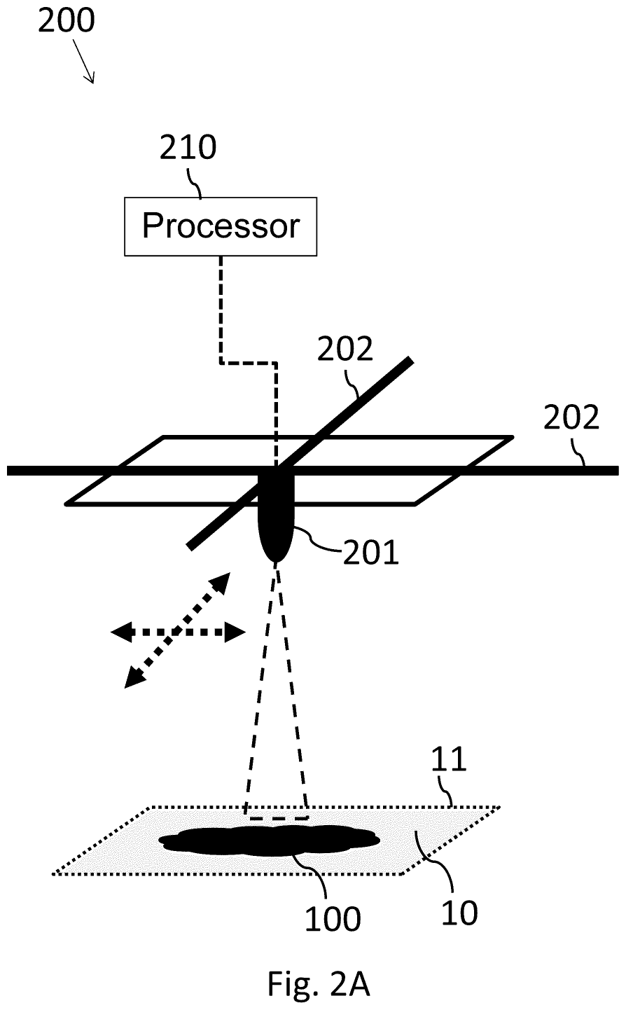

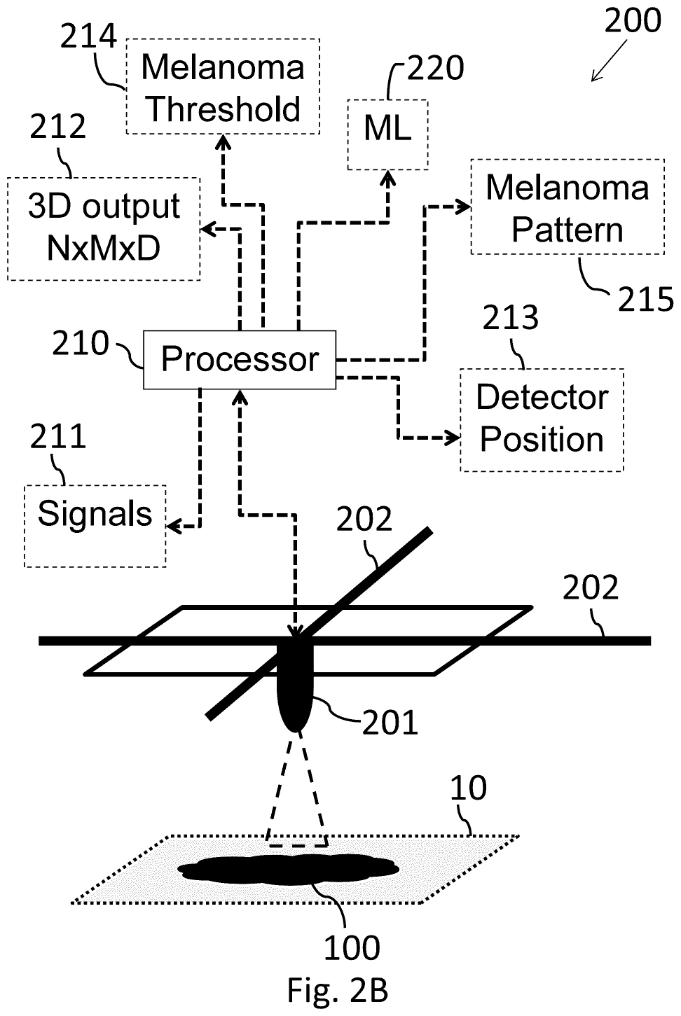

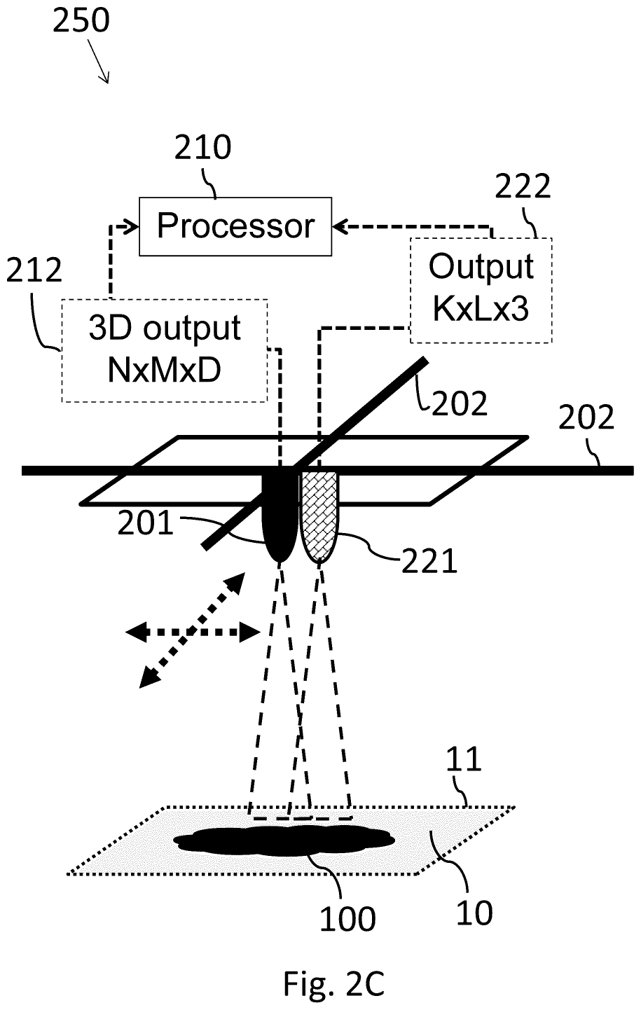

Controller 105 may be configured to execute program code to perform operations described herein. The system described herein may include one or more computing devices 100, for example, to act as the various devices or the components shown in FIG. 2. For example, communication system 200 may be or may include computing device 100 or components thereof.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of similar and/or different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein.

Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 are omitted.

For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices.

Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105.

Such a non-transitory computer readable medium may be for example a memory, a disk drive, or a USB flash memory, encoding, including or storing instructions, e.g., computer-executable instructions, which, when executed by a processor or controller, carry out methods disclosed herein.

The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPUs), a plurality of graphics processing units (GPUs), or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components.

In some embodiments, a system may include or may be, for example, a personal computer, a desktop computer, a laptop computer, a workstation, a server computer, a network device, or any other suitable computing device.

For example, a system as described herein may include one or more facility computing device 100 and one or more remote server computers in active communication with one or more facility computing device 100 such as computing device 100, and in active communication with one or more portable or mobile devices such as smartphones, tablets and the like.

Reference is now made to FIGS. 2A-2B, which schematically illustrates a system 200 for detection of melanoma 100 on a patient's skin 10, according to some embodiments of the invention. In FIG. 2B, hardware elements are indicated with a solid line, and the direction of arrows indicate a direction of information flow between the hardware elements.

The system 200 may include a detector 201 and a rail 202, configured to allow movement of the detector 201 on the rail 202 in two perpendicular axes indicated by two dashed arrows in FIG. 2A. The detector 201 may be configured to capture signals 211 that are scattered of the patient's skin 10.

The system 200 may perform spectroscopy analysis as a non-invasive measurement at the visible light range (e.g., at 300-900 nanometers wavelength) that is scattered on the borderline of epidermis and dermis layers.

For example, the detector 201 may monitor an area of the skin with 10×10 points, where each point is at a size of about 1.5 millimeter, such that areas of the patient's skin 10 are monitored to detect melanoma.

In some embodiments, the system 200 allows detection of optical and/or spatial changes in the subcutaneous layers of the patient's skin 10 that are already at the initial stage of melanoma growth, even before these changes become visible on the upper skin layer.

An early detection of the formation of a melanoma or other sterile tumors, even before it is expressed on the skin, may allow for an early medical response in a way that increases the chances of treatment and recovery.

In some embodiments, early detection of skin cancer is carried out when the optical properties (e.g., spectral reflectance in the visible range) of the tumor surface are different from those of benign tissue. These optical properties may not be constant and may change over the surface of the skin.

According to some embodiments, the detector 201 includes a light source to illuminate the skin 10 and accordingly capture the signals 211 that are scattered off the patient's skin 10.

The area scanned by the detector 201 is indicated by a dashed rectangle 11 in FIG. 2A, such that movement of the detector 201 may scan a different portion of the patient's skin 10 in order to ensure that all areas are checked. For example, the detector 201 may scan a skin area with a mole to check presence of melanoma.

For example, the movement of the detector 201 on the rail 202 may be at a resolution of 0.5 millimeters.

In some embodiments, the rail 202 is a mechanical rail, and the detector 201 is moved mechanically on the rail 202.

The system may further include a processor 210 (such as controller 105, shown in FIG. 1). The processor 210 may be coupled to or be in communication with the detector 201.

Reference is now made to FIG. 2C, which schematically illustrates a system 250 with a spectroscope 201 and dermatoscope 221 for detection of melanoma 100 on a patient's skin 10, according to some embodiments of the invention.

The detector 201 may include a spectroscope (e.g., with a substantially narrow field scan) and/or a dermatoscope (e.g., with a substantially wide field imaging to mark melanoma areas) to allow inspection of skin lesions unobstructed by skin surface reflections, where the processor 210 may be configured to perform image processing on signals received by the dermatoscope 221.

For example, the dermatoscope 221 may provide an image that is calibrated to the position of the detector 201, where each point of the skin that is scanned by the spectrometer may be represented by corresponding KxL image pixels. When the RGB spectrum is being inspected, the output 222 of the dermatoscope 221 may accordingly be provided as KxLx3.

Referring back to FIGS. 2A-2B, according to some embodiments, the processor 210 is configured to receive signals 211 captured by the detector 201, where each received signal 211 may include NxM points. The detector 201 may collect ‘D’ samples to get a three-dimensional output of NxMxD 212, where ‘N’, ‘M’ and ‘D’ are integer numbers.

The processor 210 may be configured to map the received signal 211 to the position 213 of the detector 201 on the rail 202, and to compare data from each point with a predetermined melanoma threshold 214.

In some embodiments, the processor 210 identifies at least one spread of melanoma pattern 215 based on the comparison and the mapping so as to determine if the skin 10 includes melanoma 100 based on the results of the comparison and also based on identification of the at least one spread of melanoma pattern 215.

According to some embodiments, the processor 210 is configured to train a machine learning (ML) algorithm 220 to predict presence of a melanoma 100, for instance based on pattern of the received signal 211.

The trained ML algorithm 220 may be applied, by the processor 210, on the received signals 211 captured by the detector 201. In some embodiments, the processor 210 is configured to apply clustering for input to the ML algorithm 220, where the clustering includes clustering on at least one of: patterns and spectrum responses for the received signals.

In some embodiments, clusters where the skin cells correspond to signals that are above a predetermined detection threshold indicate presence of earlier melanoma.

For example, adjacent skin cells (e.g., cells in area 11) having similar spectral characteristics may be clustered together.

According to some embodiments, the ML algorithm 220 may be trained with supervised training, for instance by medical experts.

Additional training may be carried out with a database of known positive or negative samples.

In some embodiments, the ML algorithm 220 may be trained with global training on melanoma spectrum signals for a group of all samples in the database, for example having normal reaction for positive/negative reaction to light for all data.

In some embodiments, the ML algorithm 220 may be trained with a relative training on melanoma spectrum signals for a sample of a particular patient, for example having a normal reaction for a particular patient.

In some embodiments, the clustering includes clustering on spectrum analysis compared to a reference spectrum of a sample associated with melanoma probability exceeding the predetermined melanoma threshold.

According to some embodiments, the clustering may include clustering on spectrum analysis compared to a sample of a group of patients (e.g., based on similar skin characteristics).

Thus, the system 200 allows prediction of melanoma based on the trained ML algorithm 220 and therefore increase the certainty of the predicted identification.

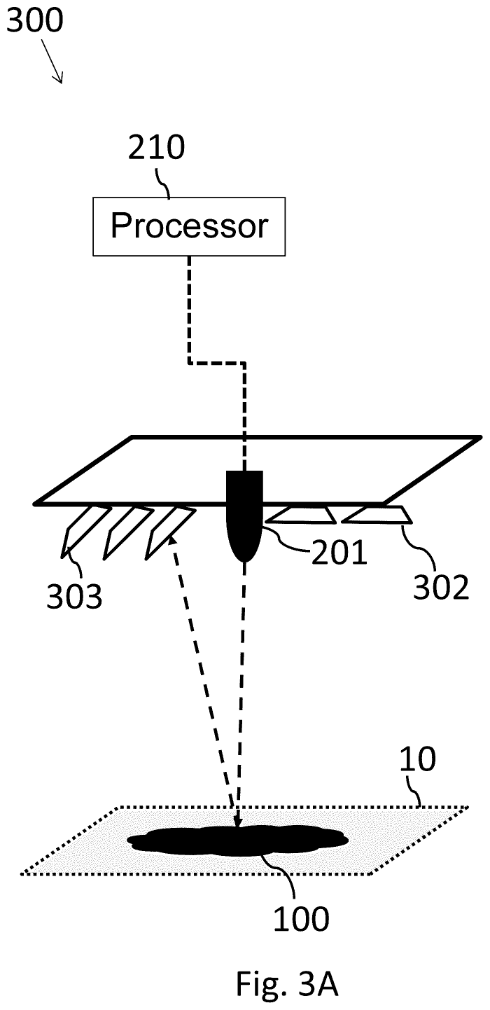

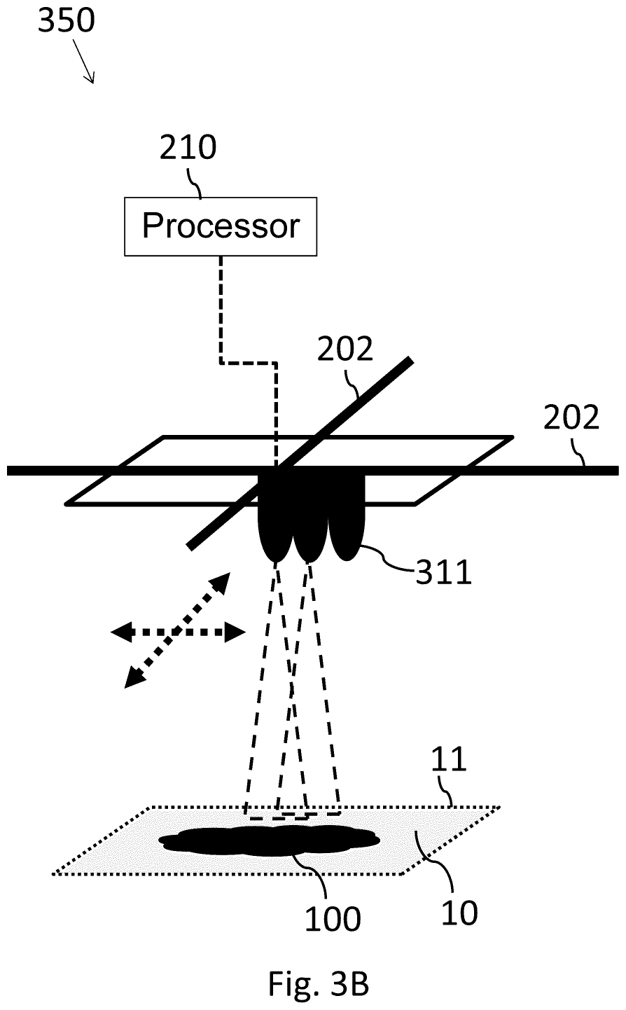

Reference is now made to FIG. 3A, which schematically illustrates a rail of a system 300 for detection of melanoma 100 on a patient's skin 10 with a mirror configuration, according to some embodiments of the invention.

According to some embodiments, the rail 202 includes a plurality of movable mirrors 302, where each mirror is movable between a first state 302 that is parallel to the rail surface and a second state 303 that is rotated by an angle as compared to the first state 302, and where the second state 303 allows receiving and sending signals to the skin.

Reference is now made to FIG. 3B, which schematically illustrates a multiple source configuration of the system 350 for detection of melanoma 100 on a patient's skin 10 with a plurality of sources irradiating the skin, according to some embodiments of the invention.

In some embodiments, the system 350 includes a plurality of sources 311 irradiating the skin 10, such that the detector receives signals from scattered irradiation.

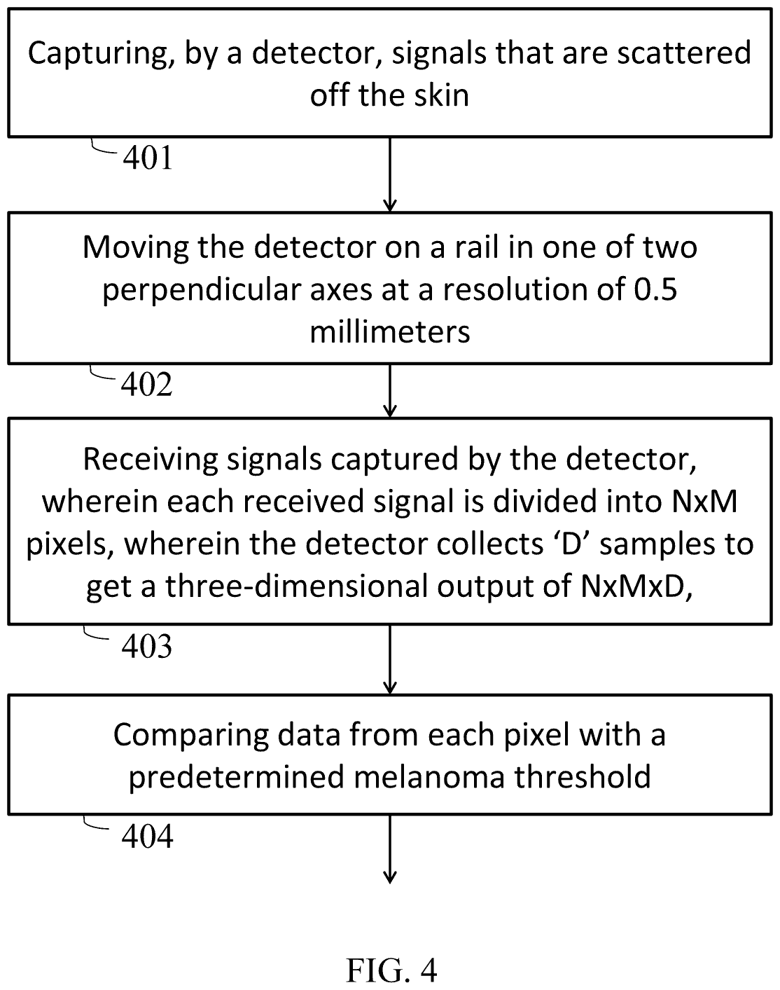

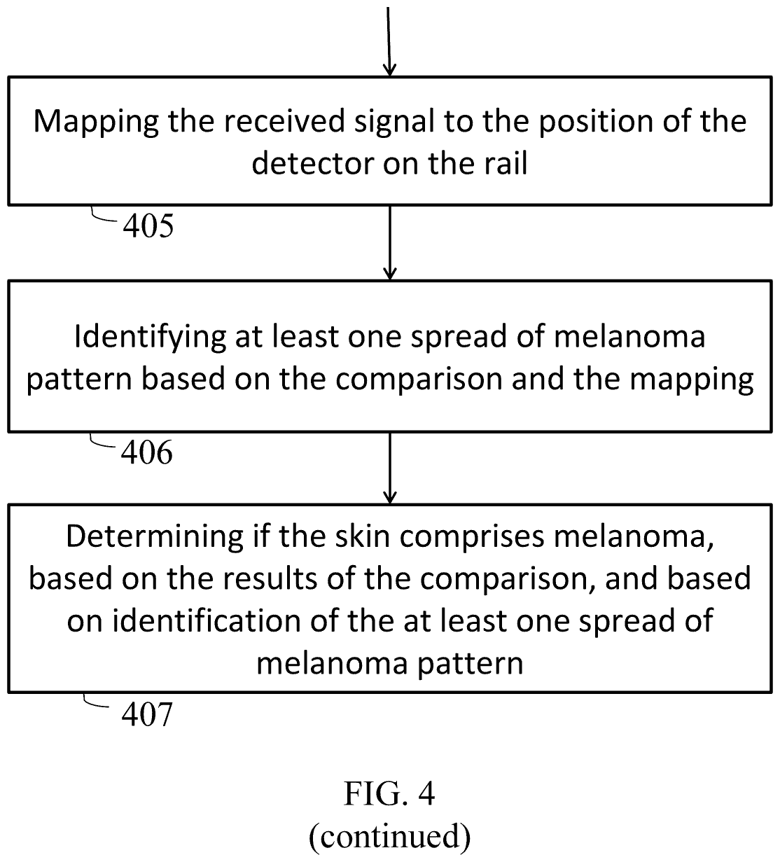

Reference is now made to FIG. 4, which shows a flowchart for a method of detecting melanoma on a patient's skin, according to some embodiments of the invention.

In Step 401, the detector captures signals that are scattered off the skin. In Step 402, the detector is moved on a rail in one of two perpendicular axes at a resolution of 0.5 millimeters.

In Step 403, captured signals are received by the detector, wherein each received signal includes NxM points, wherein the detector collects ‘D’ samples to get a three-dimensional output of NxMxD, where ‘N’, ‘M’ and ‘D’ are integer numbers.

In Step 404, data is compared from each point with a predetermined melanoma threshold.

In Step 405, the received signal is mapped to the position of the detector on the rail. In Step 406, at least one spread of melanoma pattern is identified based on the comparison and the mapping.

In Step 407, determining if the skin includes melanoma, based on the results of the comparison, and based on identification of the at least one spread of melanoma pattern.

While certain features of the invention have been illustrated and described herein, many modifications, substitutions, changes, and equivalents may occur to those skilled in the art. It is, therefore, to be understood that the appended claims are intended to cover all such modifications and changes.

Various embodiments have been presented. Each of these embodiments may of course include features from other embodiments presented, and embodiments not specifically described may include various features described herein.

1. A system for detection of melanoma on a patient's skin, the system comprising:
a detector, configured to capture signals that are scattered of the skin;
a rail, configured to allow movement of the detector on the rail in two perpendicular axes at a resolution of 0.5 millimeters; and
a processor, coupled to the detector and configured to:
receive signals captured by the detector, wherein each received signal comprises NxM points, wherein the detector collects ‘D’ samples to get a three-dimensional output of NxMxD, where ‘N’, ‘M’ and ‘D’ are integer numbers;
map the received signal to the position of the detector on the rail;
compare data from each point with a predetermined melanoma threshold; identify at least one spread of melanoma pattern based on the comparison and the mapping; and
determine if the skin comprises melanoma, based on the results of the comparison, and based on identification of the at least one spread of melanoma pattern.

2. The system of claim 1, wherein the rail is a mechanical rail, and the detector is moved mechanically.

3. The system of claim 1, wherein the rail comprises a plurality of movable mirrors, wherein each mirror is movable between a first state that is parallel to the rail surface and a second state that is rotated by an angle as compared to the first state, and wherein the second state allows receiving and sending signals to the skin.

4. The system of claim 1, further comprising a plurality of sources irradiating the skin, such that the detector receives signals from scattered irradiation.

5. The system of claim 1, wherein the detector comprises a spectroscope and a dermatoscope, and wherein the processor is further configured to perform image processing on the received signal by the dermatoscope.

6. The system of claim 1, wherein the processor is further configured to train a machine learning (ML) algorithm to predict a melanoma.

7. The system of claim 6, wherein the processor is further configured to apply the ML algorithm on the received signals captured by the detector.

8. The system of claim 6, wherein the processor is further configured to apply clustering for input to the ML algorithm, wherein the clustering comprises clustering on at least one of: patterns and spectrum responses for the received signals.

9. The system of claim 8, wherein the clustering comprises clustering on spectrum analysis compared to a reference spectrum of a sample associated with melanoma probability exceeding the predetermined melanoma threshold.

10. The system of claim 9, wherein the clustering comprises clustering on spectrum analysis compared to a sample of a group of patients.

11. A method of detecting melanoma on a patient's skin, the method comprising: capturing, by a detector, signals that are scattered of the skin;
moving the detector on a rail in one of two perpendicular axes at a resolution of 0.5 millimeters; and
receiving signals captured by the detector, wherein each received signal comprises NxM points, wherein the detector collects ‘D’ samples to get a three-dimensional output of NxMxD, where ‘N’, ‘M’ and ‘D’ are integer numbers;
comparing data from each point with a predetermined melanoma threshold;
mapping the received signal to the position of the detector on the rail;
identifying at least one spread of melanoma pattern based on the comparison and the mapping; and
determining if the skin comprises melanoma, based on the results of the comparison, and based on identification of the at least one spread of melanoma pattern.

12. The method of claim 11, wherein the rail is a mechanical rail, and the detector is moved mechanically.

13. The method of claim 11, wherein the rail comprises a plurality of movable mirrors, wherein each mirror is movable between a first state that is parallel to the rail surface and a second state that is rotated by an angle as compared to the first state, and wherein the second state allows receiving and sending signals to the skin.

14. The method of claim 11, further comprising irradiating the skin, by a plurality of sources, such that the detector receives signals from scattered irradiation.

15. The method of claim 11, wherein the detector comprises a spectroscope and a dermatoscope, and the method further comprises performing image processing on the received signal by the dermatoscope.

16. The method of claim 11, further comprising training a machine learning (ML) algorithm to predict a melanoma.

17. The method of claim 16, further comprising applying the ML algorithm on the received signals captured by the detector.

18. The method of claim 16, further comprising applying clustering for input to the ML algorithm, wherein the clustering comprises clustering on at least one of: patterns and spectrum responses for the received signals.

19. The method of claim 18, wherein the clustering comprises clustering on spectrum analysis compared to a reference spectrum of a sample associated with melanoma probability exceeding the predetermined melanoma threshold.

20. The method of claim 19, wherein the clustering comprises clustering on spectrum analysis compared to a sample of a group of patients.
